(19) United States
(12) Patent Application Publication
(10) Pub. No.: US 2011/0250679 A1
(43) Pub. Date: Oct. 13, 2011

(54) Methods and Compositions for High-Resolution Micropatterning for Cell Culture

(75) Inventor: Wesley C. Chang, Fremont, CA (US)

(73) Assignee: THE REGENTS OF THE UNIVERSITY OF CALIFORNIA, Oakland, CA (US)

(21) Appl. No.: 13/119,693

(22) PCT Filed: Oct. 1, 2009
(86) PCT No.: PCT/US2009/059194

§ 371 Date: Jun. 13, 2011

Related U.S. Application Data
(60) Provisional application No. 61/102,071, filed on Oct. 2, 2008.

(51) Int. Cl. C12N 5/079; B32B 3/00; B32B 7/12; C12N 5/071; C12N 5/02

(52) U.S. Cl. 435/325; 427/2.31; 428/343; 428/355 EN; 428/156

(57) ABSTRACT

Composite structures and methods for generating micropatterned materials suitable for use in cell culture applications are disclosed. The improvement of these compositions and methods over the prior art is based on the unexpected discovery that minor chemical modifications can be introduced to greatly enhance the adherence and/or stability of a cell-adhesive material. The micropatterned materials are inexpensive to manufacture, have long shelf-life, and are stable for prolonged periods of time under cell-culture conditions. Moreover, biologists can use these micropatterned substrates with the same ease as conventional cultureware and without the need for special sample preparation.

### CROSS REFERENCE TO RELATED APPLICATIONS

This application claims the benefit of U.S. Provisional Application No. 61/102,071, filed Oct. 2, 2008, the entire disclosure of which is hereby incorporated by reference in its entirety for all purposes.

### STATEMENT REGARDING FEDERALLY SPONSORED RESEARCH OR DEVELOPMENT

Not applicable.

### BACKGROUND OF THE INVENTION

1. Field of the Invention

The invention relates to the fields of biology, cell culture, biochemistry, and lithography.

2. Description of the Related Art

The micropatterning of cells along micron-scale features has enabled broad experimental capabilities for diverse applications in basic research, regenerative medicine, tissue engineering, as well as diagnostics and screening. See, e.g., Andersson, H.; van den Berg, A., Microtechnologies and nanotechnologies for single-cell analysis. Curr Opin Biotechnol 2004, 15, (1), 44-9, Bashir, R., BioMEMS: state-of-the-art in detection, opportunities and prospects. Adv Drug Deliv Rev 2004, 56, (11), 1565-86, Branch, D. W.; Corey, J. M.; Weyhenmeyer, J. A.; Brewer, G. J.; Wheeler, B. C., Microstamp patterns of biomolecules for high-resolution neuronal networks. Med Biol Eng Comput 1998, 36, (1), 135-41, Corey, J. M.; Feldman, E. L., Substrate patterning: an emerging technology for the study of neuronal behavior. Exp Neurol 2003, 184 Suppl 1, S89-96, Falconnet, D.; Csucs, G.; Grandin, H. M.; Textor, M., Surface engineering approaches to micropattern surfaces for cell-based assays. Biomaterials 2006, 27, (16), 3044-63, Fink, J.; Thery, M.; Azioune, A.; Dupont, R.; Chatelain, F.; Bornens, M.; Piel, M., Comparative study and improvement of current cell micro-patterning techniques. Lab Chip 2007, (advanced article), Folch, A.; Toner, M., Microengineering of cellular interactions. Annu Rev Biomed Eng 2000, 2, 227-56, and Nakanishi, J.; Takarada, T.; Yamaguchi, K.; Maeda, M., Recent advances in cell micropatterning techniques for bioanalytical and biomedical sciences. Anal Sci 2008, 24, (1), 67-72. Given these diverse benefits of cell micropatterning, there has been growing demand among researchers in academia and industry for commercial scale access to micropatterened culture substrates just as conventional cultureware has been a part of standard laboratory supply for many decades.

Indeed, there have been numerous methods pursued in recent years to prepare culture substrates with pre-defined micropatterns on which cells selectively attach to after being seeded. The introduction of microelectronic fabrication to bioengineering has provided the key technology for selectively depositing cell adhesive molecules along specific patterns with critical dimensions of microns. Micropatterning techniques include the use of photolithographic liftoff (Sorribas, H.; Padeste, C.; Tiefenauer, L., Photolithographic generation of protein micropatterns for neuron culture applications. Biomaterials 2002, 23, (3), 893-900) or a variety of “soft lithographic” techniques (Corey, J. M.; Wheeler, B. C.; Brewer, G. J., Micrometer resolution silane-based patterning of hippocampal neurons: critical variables in photoresist and laser ablation processes for substrate fabrication. IEEE Trans Biomed Eng 1996, 43, (9), 944-55, Rhee, S. W.; Taylor, A. M.; Tu, C. H.; Cribbs, D. H.; Cotman, C. W.; Jeon, N. L., Patterned cell culture inside microfluidic devices. Lab Chip 2005, 5, (1), 102-7, Wheeler, B. C.; Corey, J. M.; Brewer, G. J.; Branch, D. W., Microcontact printing for precise control of nerve cell growth in culture. J Biomech Eng 1999, 121, (1), 73-8, Whitesides, G. M.; Ostuni, E.; Takayama, S.; Jiang, X.; Ingber, D. E., Soft lithography in biology and biochemistry. Annu Rev Biomed Eng 2001, 3, 335-73), such as the popular micro-contact printing (μCP) (Wheeler, B. C.; Corey, J. M.; Brewer, G. J.; Branch, D. W., Microcontact printing for precise control of nerve cell growth in culture. J Biomech Eng 1999, 121, (1), 73-8, Chang, J. C.; Brewer, G. J.; Wheeler, B. C., A modified microstamping technique enhances poly-lysine transfer and neuronal cell patterning. Biomaterials 2003, 24, (17), 2863-70) or even direct patterning by laser ablation of molecular monolayers (Corey, J. M.; Wheeler, B. C.; Brewer, G. J., Micrometer resolution silane-based patterning of hippocampal neurons: critical variables in photoresist and laser ablation processes for substrate fabrication. IEEE Trans Biomed Eng 1996, 43, (9), 944-55, Stenger, D. A.; Hickman, J. J.; Bateman, K. E.; Ravenscroft, M. S.; Ma, W.; Pancrazio, J. J.; Shaffer, K.; Schaffner, A. E.; Cribbs, D. H.; Cotman, C. W., Microlithographic determination of axonal/dendritic polarity in cultured hippocampal neurons. J Neurosci Methods 1998, 82, (2), 167-73). To attain more effective cell patterning, a non-fouling, cell repellant material has often been deposited alongside the cell adhesive micropatterns to further enforce the compliance of cells and their processes to the desired patterns (Wheeler, B. C.; Corey, J. M.; Brewer, G. J.; Branch, D. W., Microcontact printing for precise control of nerve cell growth in culture. J Biomech Eng 1999, 121, (1), 73-8, Gombotz, W. R.; Wang, G. H.; Horbett, T. A.; Hoffman, A. S., Protein adsorption to poly(ethylene oxide) surfaces. J Biomed Mater Res 1991, 25, (12), 1547-62).

However, many current micropatterning techniques, such as those based on cell-resistant poly-ethylene-glycol (PEG) molecular monolayers, have not consistently produced high degrees of cellular compliance to desired patterns and often have difficulty producing patterns that can be maintained for more than a few days during culture. These limitations may be due to the fundamental fragility of molecular monolayers, often vulnerable to hydrolytic cleavage, as well as the difficulty in producing close-packed molecular arrangement and continuous coverage over an entire surface. Since substrates patterned with these methods must be used immediately after preparation, these techniques generally require the end-user to have knowledge and skill in surface chemistry and microfabrication and to implement the often time-consuming substrate patterning steps themselves immediately prior to preparation of cell cultures. Beyond proof-of-concept demonstrations of cell patterning, such micropatterning schemes therefore have not been successfully introduced as products widely adopted by the research community in biology despite the diverse benefits of cell micropatterning.

Low temperature deposition of robust, thin organic films via plasma-induced polymerization of monomeric precursors, considered a form of plasma-enhanced chemical vapor deposition (PE-CVD), has recently provided a new format for creating patterned cell culture (Bretagnol, F. et al., Plasma Process Polym, 3:30-28 (2006); Bretagnol, F. et al., Sensors Actuators B, 123:283-292 (2007); Bretagnol, F. et al., Acta Biomater, 2(2):165-72 (2006); Sardella, E. et al., Plasma Process Polym, 1:63-72 (2004); Goessl, A. et al., J Biomed Mater Res, 57(1):15-24 (2001); Goessl, A. et al., J Biomater Sci Polym Ed, 12(7):739-53 (2001); Forch, R. et al., Chem Vap Deposition, 13:280-294 (2007)). A key material developed for this application is a non-fouling, cell-repellant polyethylene oxide (PEO) like material, plasma polymerized from vapors of diglycol methyl ether (or any of several similar species) and deposited to fully blanket any cell culture substrate (Bretagnol, F. et al., Acta Biomater, 2(2):165-72 (2006); Mar, M. et al., Sens Actuat B, 54:125-31 (1999)). Early applications of this material used photolithographic lift-off to directly pattern the deposition of the PEO-like material (Henein, Y. et al., Sens Actuat B, 81:49-54 (2001); Pan, Y. et al., Plasma Polymers, 7(2):171-183 (2002)). However, the PEO-like material has also been used as a blanket cell repellant foundation on which bioactive species were introduced via μCP (Henein, Y. et al., Sens Actuat B, 81:49-54 (2001); Pan, Y. et al., Plasma Polymers, 7(2):171-83 (2002); Ruiz, A. et al., Microelectr Engin, 84:1733-1736 (2007)) or on which other types of organic films—varieties that promote cell attachment—were patterned (Bretagnol, F. et al., Plasma Process Polym, 3:30-8 (2006); Sardella, E. et al., Plasma Process Polym, 1:63-72 (2004)). Subsequent work introduced the concept of “tuning” or selectively altering the surface properties of the PEO-like film itself to render it cell adhesive only on the desired areas. For example, applications of microwave-generated Ar/H₂ plasma (Bretagnol, F. et al., Sensors Actuators B, 123:283-292 (2007)) or electron beam lithography (Bretagnol, F. et al.; Nanotech, 19:125306 (2008)) have been used to tune the PEO-like character and the surface topography to render specific regions cell adhesive, while leaving adjacent areas cell repellant.

One unaddressed barrier to enhanced research productivity using neuronal cell cultures is the disorganized distribution and random arrangement of neurons and their axons in conventional, unpatterned culture dishes. While neuroscientists have developed imaging and image processing capabilities to improve experimental throughput, many of these solutions are expensive to implement and do not directly address the challenges of locating and distinguishing individual neurons in a disorganized culture.

Thus, there remains a need in the art for a low cost, user-friendly cell culture product that contains robust micropatterns to organize cells into specific micropatterns for a wide variety of cell-based applications. The present invention addresses these and other shortcomings of the art by providing micropatterned cultureware that enables effective control over the positioning, orientation, and shape of individual cells for study and enhanced experimental throughput and cell-based screening capabilities, that is inexpensive to manufacture, easy to use, and storable in a laboratory setting, and that is compatible with standard cell culture protocols without need for additional preparation by the user. Furthermore, the present invention enables cultured cells to take hold and develop on the substrate, advantageously allowing the desired micropatterns to persist and permitting the cells to survive and develop within desired micropatterns for extended durations on the order of several weeks.

### SUMMARY OF THE INVENTION

Embodiments of the invention are directed to methods for producing a new type of reliable, low-cost cell culture platform for precisely organizing cells into patterned arrays to enable high-content and high-throughput assays of cell function in vitro.

While the specific embodiments described herein are directed to neuronal culture, embodiments of the invention can extend to other cell types and applications that benefit from organizing cells into neat arrays according to predetermined patterns.

Accordingly, in one aspect, the invention provides a method comprising depositing a cell-repellant film on a substrate, masking a region of the cell-repellant film or substrate, modifying the masked region, and depositing a cell-adhesive material on the modified region.

In one aspect the cell-repellant film is masked. In another aspect the substrate is masked. In yet another aspect, the mask is a photolithographic mask. In still another aspect, the cell-repellant film is deposited using a plasma-enhanced chemical vapor deposition process. In still another aspect, a polypeptide is adsorbed onto the cell-adhesive material. In certain aspects, the polypeptide is an immunoglobulin, a serum albumin, or a laminin.

In another aspect, cells are deposited on the deposited cell-adhesive material. In another aspect, the cell-adhesive material is deposited and patterned before deposition of the cells. In other aspects, the cells are fibroblasts, retinal ganglion cells, hippocampal neurons, or a combination thereof.

In yet another aspect, the cell-repellant film comprises CH3-O-(CH2-CH2-O)n-CH3, wherein n is an integer from 1 to 7. In another aspect, the modifying step comprises exposing the cell-repellant film to an oxidizing agent. In another aspect, the oxidizing agent is an oxygen plasma.

The invention also provides a composite structure comprising a substrate, a cell-repellant film deposited on the substrate, wherein one or both of the cell-repellant film and the substrate comprise a modified region, and a cell-adhesive material adsorbed to the modified region. In one aspect, the cell-repellant film comprises CH3-O-(CH2-CH2-O)n-CH3, wherein n is an integer from 1 to 7. In another aspect, the cell-repellant film is produced using a plasma-enhanced chemical vapor deposition process. In still another aspect, the modified region of the cell-repellant film comprises a chemical modification caused by exposure to an oxidizing agent. In certain embodiments, oxidizing agent is an oxygen plasma. In certain embodiments, the chemical modification comprises the presence of a carboxylate group, an ester group or combinations thereof.

In yet another aspect, the cell-adhesive material is a monolayer physisorbed onto the modified region of the cell-repellant film. In another aspect, the cell-adhesive material comprises a polycationic molecule. In another aspect, the polycationic molecule is poly-lysine or polyornithine.

In another aspect a polypeptide is adsorbed to the cell-adhesive material. In certain embodiments, the polypeptide is an immunoglobulin, a serum albumin or a laminin.

In another aspect, the cell-adhesive material comprises a predetermined pattern of features. In certain embodiments, the predetermined pattern of features comprises feature elements having a dimension in the range of 1 μm to 100 μm, while in other embodiments, the feature elements have a dimension in the range of 1 μm to 10 μm, and in still other embodiments, the feature elements have a dimension in the range of 1 μm to 5 μm.

In another aspect, the invention provides a composite structure as described above, further comprising cells adherent to the cell-adhesive material. In certain embodiments, the cells comprise neurons. In other embodiments, the neuronal cells form a synapse. In still other embodiments, the synapse is formed at a predetermined location. In other embodiments, the cells comprise fibroblasts, retinal ganglion cells, or hippocampal neurons.

In yet other embodiments, the invention provides a stable composite structure wherein the predetermined pattern of features is stable for at least two months when stored at 20° C. and 50% relative humidity. In yet other embodiments, the invention provides a stable composite structure wherein the predetermined pattern of features is stable for at least twenty-one days when held at 37° C. and immersed in a cell-culture medium.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 μ-Poly-Lysine-Adsorption-on-Cell-Repellant (μPLACeR) patterning process. (A) Process layout. (B) Resolution test patterns (numbers indicate pattern size in microns).

FIG. 2 High resolution XPS analysis of the film surface used to quantify the proportion of various types of carbon bonding within the PEO-like material; the C1 (carbon) peak has four main contributions: at 285 eV(C₁), 286.5 eV(C₂), 288 eV(C₃), and 289.2 eV(C₄) corresponding to the different types of chemical bonds involving carbon.

FIG. 3 AFM imaging of the surface topography of the native film's surface; (A), a topographical mapping of a 5×5 μm region; (B) Representative linear trace across surface before (upper trace) and after (lower trace) brief plasma oxidation.

FIG. 4 (A) Adsorption of molecular species from aqueous solution onto PEO-like film, both native and oxygen plasma treated. Bold solid line indicated the level of adsorption on cell culture glass. (Vertical scale is arbitrary units.) (B) The level of adsorbed poly-lysine retained before and after photoresist stripping process for both native PEO-like film and oxygen plasma treated film.

FIG. 5 (A) Cell (Hippocampal neuron) viability and compliance was evaluated on a patterned checkerboard with 140 μm squares. Fluorescently labeled poly-lysine was used to mark the cell adhesive squares, where cell bodies attached and appear as a lighter background compared to the bare PEO-like film, the cell-repellant regions. Viable cells have been labeled with Fluo-4 calcium indicator. Cell attachment is exceedingly rare on the adjacent areas containing bare PEO-like film. (None were encountered in this sampled region). (B) Along edges of cell adhesive regions, a local increase in cell density is typically seen. This effect is possibly due to limited migration of neuronal cell bodies away from cell repellent regions towards the cell adhesive regions, although a definitive explanation for this effect remains to be determined. (scale bar=200 μm) (C) An array of 70 μm-wide, circular cell adhesive regions interconnected by narrow, 2 μm-wide, 200 μm-long cell adhesive lanes. The circular regions supported the attachment and growth of neuronal cell bodies, while the interconnecting lanes served as conduits to direct the outgrowths of neurites. Compliance of both cell body attachment and neurite outgrowth was nearly perfect on these patterns.

FIG. 6 Schematic illustration of “piggybacking” embodiment in which cell-adhesive material such as, e.g., poly-lysine is used as an intermediate capture agent for another cell-adhesion molecule such as a polypeptide or protein (e.g., BSA, laminin, immunoglobulin, etc.).

FIG. 7 The process of poly-lysine deposition on PEO-like film was used to produce micropatterns of various shapes and configurations for neuronal cell body attachment and neurite outgrowth. In (A), straight lanes of 10 and 20 micron widths permitted neuronal cell bodies to attach as well as neurites to take hold and extend. Due to the proximity of the cell adhesive lanes, neurites can sometimes cross the cell repellent areas to make connections with neurites and cells on nearby lanes. (B) A grid pattern with “wells” (circular, cell adhesive regions, 70 μm dia.) and interconnecting lanes (200 μm long, 2 μm wide) were used to test the compliance of both the cell bodies and neurites. Cell bodies remained exclusively within the wells, while neurites extending from these neurons followed the narrow lanes. (C) A wider field of view (Upper: bright field, Lower: fluorescent) of a series of wells connected by 200 μm long channels shows cell bodies restricted to the circular wells while neurites run faithfully within the channels. (D) Tubulin within axons extending on micropattern substrates can be visualized after using an anti-tubulin antibody and standard cellular immunolabeling methods and observation using conventional fluorescence optical imaging. (Upper: brightfield; Lower: fluorescent) In (E), a neurite following the contours of a circuitous lane can be seen. However, at sharp turns, the neurite can be seen to “cut corners” (arrow). The adhesive lane extends along the dotted line to the cell adhesive patch at right. However, the neurite, which originates from the adhesive patch, cuts this corner. Except for cutting sharp corners, neurites were highly compliant with the patterned lanes. (scale bar=50 μm) (F) Micropatterned substrates stored for over 1 month in room temperature and atmosphere conditions remained bioactive, permitted highly viable cultures, and produced a high degree of cellular compliance similar to that of substrates used soon after production (circular, cell adhesive regions, 70 μm dia.; lanes 200 μm long, 2 μm wide). (G) Example of molecular ‘piggyback’ in which poly-lysine is used to further immobilize the extracellular matrix molecule laminin. The successful micropatterning of laminin using this method was verified by analyzing the pattern of neurite outgrowth from retinal ganglion cells (RGC), which is known to be laminin dependent, and do not extend on poly-lysine alone. The neurites of RGCs were found to follow faithfully the original poly-lysine pattern (scale bar=200 μm).

FIG. 8 shows cross-sections of precursors used to form patterned substrates according to another embodiment of the invention. A modification of the process shown in FIG. 1 is shown in Step 4 in FIG. 8. Instead of simply treating with brief oxygen plasma, the part of the film revealed by the photolithographic development is etched away to expose the underlying glass substrate. The etching of the film can be accomplished by exposure to ionized gases. Following the etching of the film, the exposed glass is treated with the brief oxygen plasma to assist in the adsorption of poly-lysine.

FIG. 9 shows cultured 3T3 fibroblasts using micropatterned substrates having a variety of test patterns (FIG. 9A-C), and brightfield and fluorescence microscopy (FIG. 9D, E).

FIG. 10 shows long term culture results for neurons following 23 days of culture (micrograph, FIG. 10A) on patterned on substrates according to the present invention (Fig. micropattern schematic with dark areas cell adhesive, FIG. 10B).

### DETAILED DESCRIPTION OF THE INVENTION

Advantages and Utility

The fabrication can be performed in batch formats, which permits multiple copies of a desired micropattern to be simultaneously produced with high yield. This ease of manufacturing translates into low unit costs, which in turn allows the technology to be applied to produce single-use, disposable devices. The stability of the micropatterned substrate also enables long shelf-life without degradation in function as well as longevity of micropatterns during cell culture. These two aspects of cultureware longevity are key requirements for experimental biologists and have thus far represented a major barrier for conventional micropatterning methods.

The technique does not require complex chemistries, and the resulting patterned film has extended shelf-life in ambient air. Additionally, this process is compatible with standard microfabrication processes and therefore, cellular and subcellular scale micropatterns can be integrated with virtually any biosensor and microdevice.

For our technology, the numerous advantages it has over the prior art enables it to suitably serve as the basis for cheaply producing user-friendly cultureware that reliably provides micropatterning of cell culture with high compliance and longevity. Biologists can use these micropatterned substrates with the same ease as conventional cell cultureware and will not require special skills or sample preparation.

### DEFINITIONS

Terms used in the claims and specification are defined as set forth below unless otherwise specified.

Abbreviations used in this application include the following:

Adsorbed means molecularly associated with and is intended to encompass covalent and non-covalent interactions.

AFM means atomic force microscopy.

APTES means aminopropyltriethoxysilane.

BSA means bovine serum albumin.

DETA means diethylenetriamine-propyltrimethyoxysilane.

DI means deionized.

Diglyme means diglycol methyl ether (CAS number 111-96-6).

HMDS means hexamethyldisilazane.

LP-CVD means low pressure chemical vapor deposition.

NHS means N-hydroxysuccinimide.

PBS means phosphate buffered saline.

PEO means polyethylene oxide.

RGC means retinal ganglion cell.

SAM means self-assembled monolayer.

T means Torr.

XPS means X-ray photoelectron spectroscopy.

It must be noted that, as used in the specification and the appended claims, the singular forms “a,” “an” and “the” include plural referents unless the context clearly dictates otherwise. Any recitation of “or” in the claims should be interpreted so as to provide the broadest valid claim construction. In some instances, “or” may be construed to mean “and/or.”

Embodiments of the invention include a novel extension of the use of PEO-like films. The PEO-like film preferably comprises CH3-O-(CH2-CH2-O)n-CH3, where n is an integer from 1 to 7. In certain embodiments, n is an integer from 2 to 5, or n is an integer from 2 to 4. This material, even though it is a highly “non-fouling” form, is in fact capable of modestly adsorbing from aqueous solution a polycationic species such as, e.g., poly-lysine, a positively charged polypeptide that promotes cell adhesion. This discovery was unexpected and has served as the basis for the invention, which provides significant advantages over the prior art. This adsorption is further enhanced with slight chemical alteration of the surface chemistry via, e.g., exposure to an oxidizing agent such as, e.g., a brief plasma oxidation. This treatment represents a more subtle method for tuning surface properties than previous modifications demonstrated for this PEO-like film.

Furthermore, oxidation such as, e.g., plasma oxidation, when combined with the adsorption of a poly-cationic species such as, e.g., poly-lysine or polyornithine, can be leveraged not only to enable direct cell attachment but also to mediate the immobilization of other molecular species that could not otherwise be immobilized to the surface of the film without chemical derivatization. In certain embodiments these species are polypeptides, such as, e.g., immunoglobulins, serum albumins, or laminins.

Embodiments of the invention have harnessed the interaction of polycationic species such as, e.g., poly-lysine with PEO-like films to develop a simple and yet versatile and high-resolution micropatterning scheme that uses only a single deposition of a blanket background of PEO-like film along with a single microlithographic step to create micron-scale adhesive regions to effectively restrict the regions where deposited cells anchor and grow under cell culture conditions.

Exemplary cells include fibroblasts, retinal ganglion cells, or hippocampal neurons, although other cell types can be used, including myocytes, myoblasts, endocrine cells, neurendocrine cells, paracrine cells, and any other cell type that can be advantageously cultured under conditions restricting the organization of the cultured cells. In certain preferred embodiments, the cells are neurons, and the micropatterning scheme is used to control body attachment points to the micropatterned surface and to strictly guide axon growth.

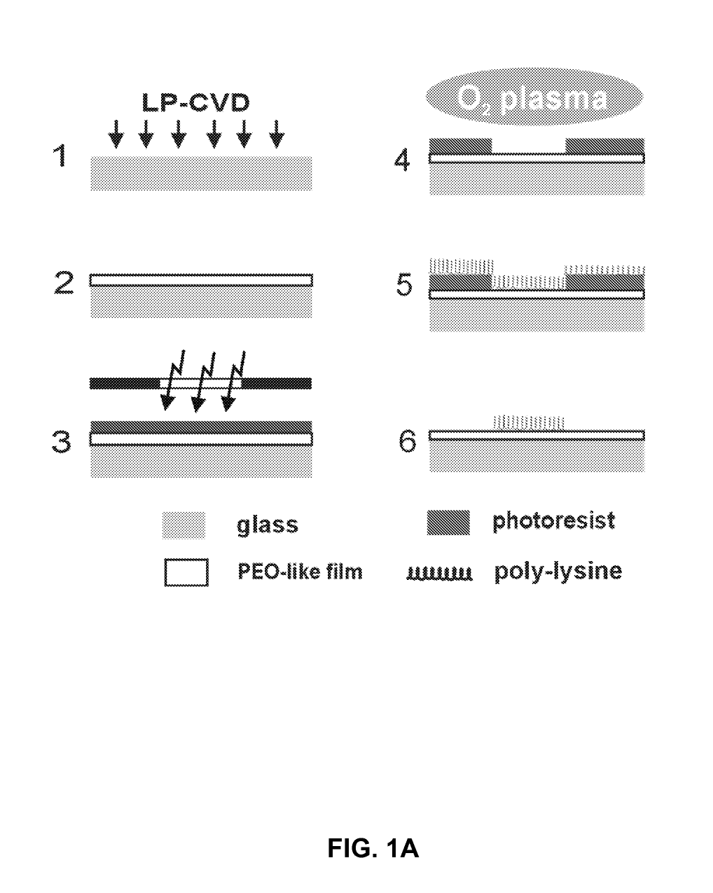

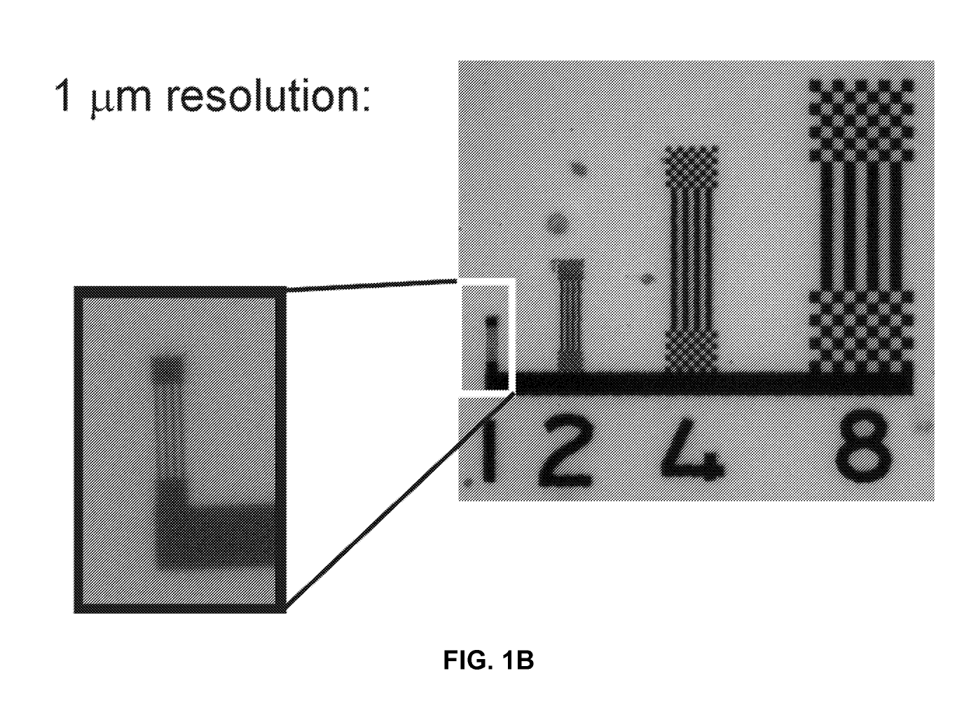

Features of embodiments of the invention can be described with respect to FIG. 1. Novel features associated with the process shown in FIG. 1 include: (1) the use of a cell repellant background (in this case, a plasma polymerized, PEO-like material), parts of which are later rendered cell adhesive; (2) subtle chemical modification (via, e.g., oxygen plasma treatment) of the material's surface to render it more receptive to molecular adsorptions (Step 4); and (3) the immobilization of a cell-adhesive molecule (such as, e.g., poly-lysine) to the modified surface (Step 5); and the cell-adhesive molecule can also be used to mediate the immobilization of other cell-adhesive or bioactive molecules.

Embodiments of the invention are not limited to the specific embodiments described above.

Although PEO-like files are described in detail above, embodiments of the invention may include other types of cell repellant films. These alternative materials can include any of a variety of plasma polymerized films, including fluorinated, “Teflon-like” materials. Additionally, more conventional surface coatings may also be used as the cell repellant background film. These include (but are not limited to) a variety of polymer materials that can be “spin cast” onto a planar substrate. (One example is the Cytop, “Teflon-like” coating that is “spin cast” onto surfaces).

Also, in step 4 of FIG. 1, an oxygen plasma treatment is used to chemically modify the surface in order to render it more receptive to protein and molecular adsorption. The oxygen plasma contains ionic species that chemical react with the surface. One desirable aspect of this is that the treatment produces an increase in the density of hydroxyl, carboxylate, and ester groups on the surface. This surface modification, however, can also be brought about by other treatments, including immersion in basic solution or in hydrogen peroxide, or exposure to ultraviolet (UV) light.

While poly lysine was used as the cell adhesive molecule in the specific examples described above, in principle, any positively charged, polymeric peptide can be used in place of poly-lysine. For example, polyornithine is an alternative, since its behavior is very similar to that of poly-lysine, and is positively charged at neutral pH. Beyond this, there are many other cell adhesive or bioactive molecules can be applied to the modified surface and can be immobilized via surface adsorption. Examples include collagen, fibronectin, and gelatin.

In addition, covalent immobilization can be used as well, instead of adsorption. Covalent attachment via a silane linking group can be especially well suited to attach cell-adhesive groups to the surface. Silane linker groups in particular can benefit from the addition of —OH species on the surface. For example, aminopropyltriethoxysilane (APTES) is commonly used to form a self-assembled monolayer (SAM) on surfaces to render them cell adhesive. The SAM formed from APTES can be used in place of poly-lysine adsorption. Of course, APTES is just one example of silane-linked molecules that can be used.

Functional groups can be advantageously used in the practice of the invention. As used herein, a functional group can be either a group that by itself confers cell adhesive properties (for example, positive charge) or more generally can be used as an intermediary to link with other bioactive molecules (usually proteins). Some examples include amino silanes, (amine group as “functional group”), such as Aminopropyltriethoxysilane (APTES, or APTS) and Diethylenetriamine-propyltrimethyoxysilane (DETA). Linker molecules may be functionalized with: (“functional groups”) N-hydroxysuccinimide (NHS), aldeyhyde, maleimide, vinyl sulfone, pyridyil disulfide, epoxies such as 3-glycidoxoypropyl-trimethoxysilane (3-GPS), etc. Within this realm, there are numerous combinations of functionalized linker molecules that can be used.

The Examples below describe the results of specific tests conducted to evaluate the effectiveness and versatility of the patterning technique. The tests can: 1) compare the adsorption of a few key molecular species on the PEO-like film; 2) demonstrate the ability of an immobilized polycationic species such as, e.g., poly-lysine to mediate the adsorption of these other species; 3) assess the viability of primary neurons and their ability for neurite outgrowth on patterned PEO-like films; 4) quantify the compliance of cultured neurons and their axons with respect to the cell adhesive and adjacent cell repellant patterns; and 5) determine whether photolithographic processes resulted in any chemical changes to the surface of the PEO-like film.

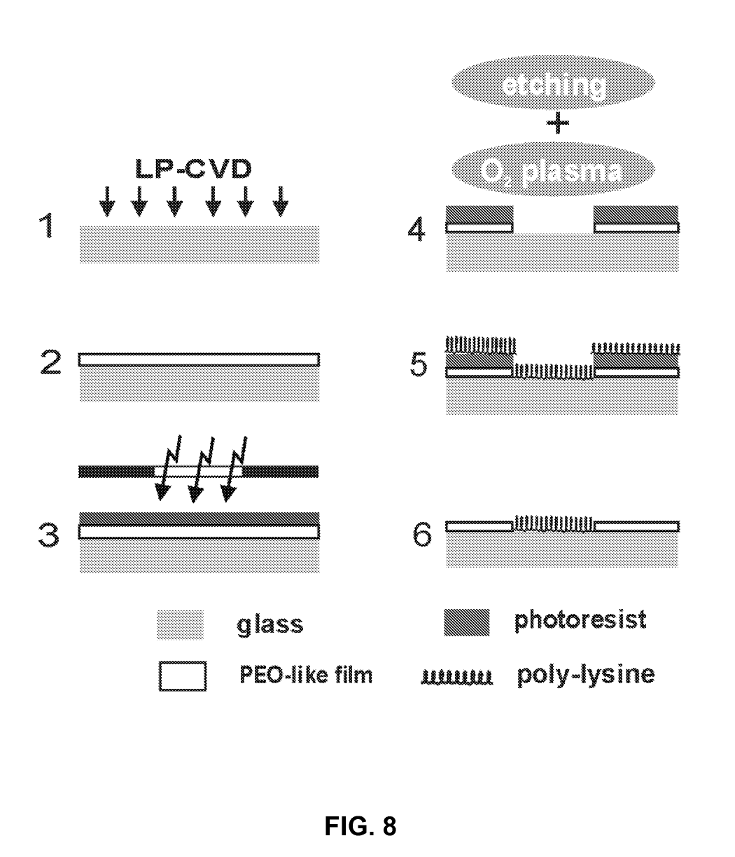

A viable modification to the fabrication process can be inserted Step 4 is shown in FIG. 8. Instead of merely modifying the exposed surfaces of the polymeric, PEO-like film (as in the original process), an alternative step is to etch away the polymeric film in those exposed areas, revealing the underlying substrate (e.g., glass). The removal of this material can be accomplished by either dry plasma etching or wet chemical treatment. The revealed substrate can in turn be further modified via oxidation and/or plasma treatment to enhance the adsorption of cell-adhesive material as well as the actual attachment of cells in culture. In the example illustrated in FIG. 8, Step 5, poly-lysine is deposited via physisorption to the surface following the etching of the polymeric film and surface modification of the underlying areas. However, as in the process illustrated in FIG. 1A, numerous other materials and molecules can be substituted for poly-lysine in bringing about a cell adhesive surface. Then, in the final step (transition between 5 and 6 in FIG. 8), the photoresist is stripped away, leaving a micropatterned substrate in which the cell-adhesive areas have cell-attachment promoting material directly immobilized on substrate, while the cell-repellant areas still have the unmodified, PEO-like polymer. This final composite product can be used in the same fashion as the alternative micropatterned substrates (having cell-adhesive areas attached to modified regions of cell-repellant area as illustrated in panel 6 of FIG. 1A) and can also be used for the same applications involving cell culture. An advantage of using this variation of the micropatterning process is that cultured cells adhere to a surface that is more akin to conventional culture substrate (e.g., glass plus cell attachment molecules). Also, this micropatterned substrate can likewise be used to “piggyback” other bioactive molecules selectively along the micropatterns, as described above.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Process Overview

This example provides an overview of a process for creating used to create poly-lysine micropatterns on the surface of a glass substrate. The glass substrate (usually a 4″ Pyrex wafer (Pyrex 7740, double-side polished, University Wafer, Boston, Mass.) was positioned on the lower, ground electrode of a parallel plate plasma system. As diagrammed in FIG. 1A(1), process gas, comprising 20% vapors of diglycol methyl ether (CAS#111-96-6, J. T. Baker, Phillipsburg, N.J.)] (“diglyme”) in argon (Ar) was introduced into the chamber at a total pressure of ˜20 mT. An RF generator (Plasma-Therm PK-12, Plasmatherm LLC, St. Petersburg, Fla.) was used to induce a plasma using a power of approximately 1-2 W. Under these conditions, the diglyme molecules polymerized to form a PEO-like, solid material that deposited uniformly on the glass substrate as shown in FIG. 1A(2). The substrate, after being blanketed with the PEO-like film then underwent standard photolithography. Photoresist (OiR 10i) (Arch Chemicals, Norwalk, Conn.) was spin coated onto the surface of the PEO-like film and then exposed by UV through a photomask containing the desired micropatterns as shown in FIG. 1A(3). After the exposed photoresist was developed, the underlying film was opened in the UV exposed regions, while photoresist remained to cover the adjacent areas. The surface was then briefly treated with oxygen plasma to chemically modify the exposed areas of the film as shown in FIG. 1A(4). This was followed immediately by incubation with poly-lysine solution to immobilize this molecule on the film surface as shown in FIG. 1A(5). According to known “lift-off” patterning techniques, the remaining photoresist was then removed as shown in FIG. 1A(6), leaving poly-lysine only in the desired regions to promote cell adhesion. The adjacent regions of the PEO-like film, which were protected by photoresist, preserved their non-fouling character and remained cell repellant. Thus, a cell adhesive pattern is surrounded by a stable, cell repellant surface. FIG. 1B illustrates patterns produced using this method, having features with dimensions on the order of 1 μm. Additional process details are provided below.

PEO-like film deposition. A film was deposited in a Plasma-Therm PK-12 (Plasmatherm LLC, St. Petersburg, Fla.), parallel-plate plasma system using platens approximately 12 inches in diameter. During deposition, a mixture of 20% diglycol methyl ether ((CH₃OCH₂CH₂)₂O, or DEGDME, or “diglyme”) (CAS #111-96-6, J. T. Baker, Phillipsburg, N.J.) vapor in argon (Ar) was maintained in the chamber at a total pressure of ˜20 mT. An RF generator (operating at 13.56 MHz) produced plasma at a constant power of ˜1-2 W in a low temperature environment (approximately 25° C.). Deposition was performed for about 20 min. on cleaned, polished Pyrex glass, positioned on the lower, ground electrode.

### Example 2

### Oxygen Plasma

Oxygen plasma. Pyrex samples with deposited film were treated with oxygen plasma using a March Plasmod plasma system (March Plasma System, Concord, Calif.). Surfaces were treated at 25° C. with 20 W of oxygen plasma for 15 sec. at ˜1.3 T. The duration of the oxygen plasma was limited to avoid eroding the photoresist and distorting the lithographic pattern.

### Example 3

### Contact Angle

Contact angle. The wetability of water on the PEO-like film was measured using a Kruss Contact Angle Measuring System (Kruss GmbH, Hamburg, Germany). Contact angles were determined from magnified images of sessile drops of ˜10 μL deposited on the film surface with a miniature syringe. Numerous drops were measured for each sample, and data represent an average of at least 10 measurements.

### Example 4

### XPS Analysis

XPS analysis. X-ray photoelectron spectroscopy was performed by an SSI S-Probe Monochromatized XPS Spectrometer with a monochromatic Al Kα X-ray small spot source (1486.6 eV) and a take off angle of 45°. For characterization of film composition, a broad survey spectrum (0-1000 eV) was performed spot size of 1000×250 μm. This broad spectrum permitted the quantification of the relative surface compositions of C and O species based on the C1 and O1 peaks. Additionally, high-resolution spectra using a spot size of 800×150 μm were also compiled for the 278 to 294 eV range to elucidate the relative contributions from the C1 peak's individual components, which represented signals from carbon bonding with different atomic species. For each high resolution spectrum, the individual components were determined from fitting the total spectrum to known peaks at 285, 286.5, 288, and 289.2 eV using Gaussian-Lorentzian fitting (XPSPEAK 4.1). To prevent interference from chemical species in the underlying substrate, film thickness deposited on samples used in the XPS exceeded 30 nm, so that all measurements were from molecules from the film. (The X-ray source from XPS penetrated the material to a depth of about 10-20 nm from the surface.)

Poly-lysine Micropatterned PEO-like films after photolithography. Films that had undergone the entire photolithographic process, from photoresist application to development and stripping were characterized using XPS to determine whether these treatments altered the chemical composition of the underlying material. (On samples for XPS analysis, the poly-lysine was not introduced to the surface.) In addition, the degree to which poly-lysine that was adsorbed to the PEO-like film withstood the photoresist stripping process was investigated by comparing the binding of fluorescently-labeled poly-L-lysine (Sigma-Aldrich, St. Louis, Mo.) to the film surface before and after stripping. Of interest was whether and to what extent the photoresist stripping treatment removed adsorbed poly-lysine.

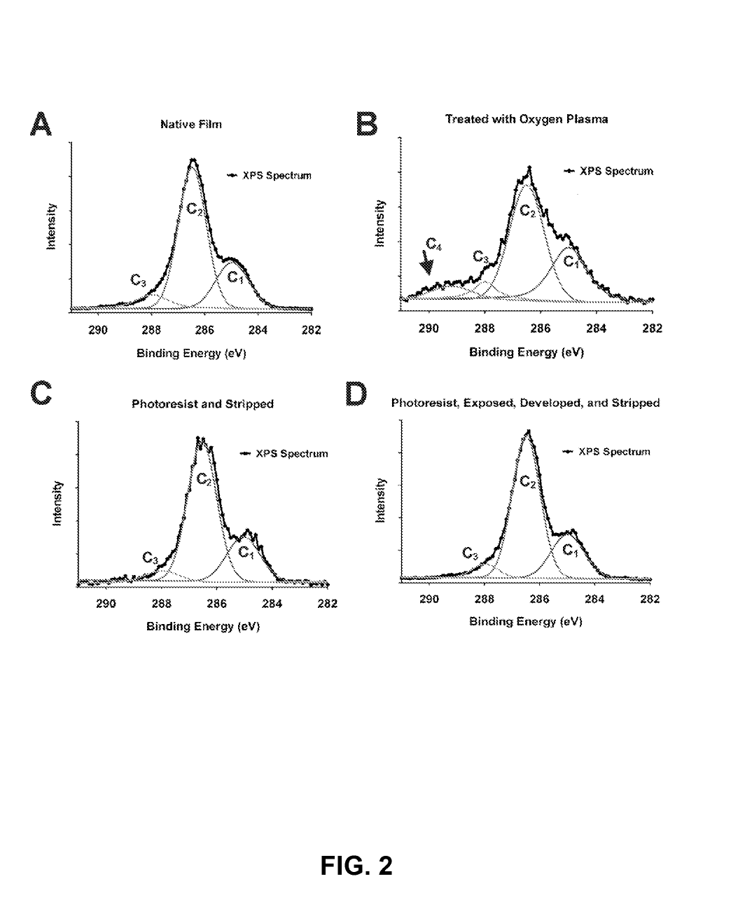

The native film, as deposited, contained a stoichiometric ratio of oxygen to carbon (O/C) of approximately 0.5. From the high resolution spectrum, FIG. 2(A), the PEO-like character was about 70%, given the ratio of C—O to C—C/C—H bonds. This film was found to be highly non-fouling and cell repellent. With brief plasma oxidation, FIG. 2(B), PEO character was diminished somewhat to about 55%, while the presence of ester and carboxyl (COOR/H groups) increased markedly (arrow). During photolithography, the native film was subjected to various solvent treatments. In FIG. 2(C), native film was subjected to HMDS treatment, photoresist coating and then stripping. This is the treatment that unexposed film is subjected to during the photolithography. To represent what happens to the film underneath regions where photoresist was exposed and subsequently developed away, in FIG. 2(D) the film was subjected to HMDS treatment, photoresist spin coating, UV exposure, treatment with developer solvent and photoresist stripping at the end. In the experiments illustrated in both FIGS. 2(C) and 2(D), the photolithographic processes did not alter the chemical composition of the film or the relative proportion of carbon-based chemical bonds.

### Example 5

### AFM Film Characterization

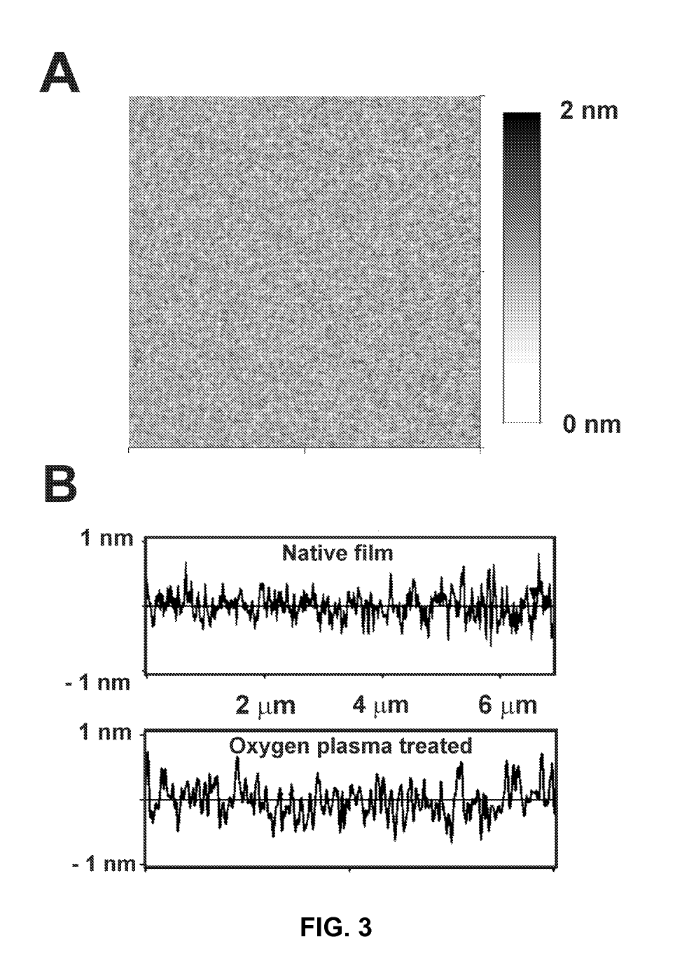

AFM film characterization. A Digital Instruments (Veeco, Plainview, N.Y.) Nanoscope Dimension 3100 atomic force microscope was used with a cantilever probe in tapping mode to characterize the topography of the film surface and to determine film thickness via step height measurement. As shown in FIG. 3A, a topographical mapping of a 5×5 μm region shows that the surface roughness remains within a 2 nm range. This is also shown in an arbitrary (but representative) linear trace across the film surface before (FIG. 3B, upper trace), and after brief plasma oxidation (FIG. 3B, lower trace). The degree of roughness was unchanged even after the brief plasma oxidation (B, lower).

### Example 6

### Protein Adsorption

Protein adsorption. To quantify the adsorption of protein (Chang, T. Y. et al., Langmuir, 23(23):11718-25 (2007)), phosphate buffered saline (PBS) solutions (pH=7.2) containing: 1) fluorescein-labeled poly-lysine (200 μg/mL), or 2) bovine serum albumin (BSA) (100 μg/mL), or 3) immunoglobulin G (IgG) (100 μg/mL) were incubated on both native and oxygen plasma-treated PEO-like film for 1 hour each at room temperature conditions. After the incubation, the samples were washed with DI water and air-dried. In addition, the ability of pre-adsorbed poly-lysine to immobilize IgG was determined in samples that were first incubated for 1 hr with 200 μg/mL of unlabeled poly-lysine, washed and dried, followed by incubation of 100 μg/mL of fluorescein-labeled IgG for an additional 1 hour. To provide a point of comparison, the adsorption of each of species (poly-lysine, BSA, and IgG) was also performed on bare cell culture glass (MatTek Cultureware, MatTek, Ashland, Mass.). The level of fluorescence present on the substrate (both PEO-like film and plain glass) following the various incubations was quantified by observation under a standard inverted microscope (Nikon TE 2000) under 10× objective magnification using a FITC filter and illuminated by a 150 W Hg lamp (Optiquip, Highland Mills, N.Y.). Images were collected via a Retiga Q-Imaging Exi (Q-Imaging, Surrey, BC Canada), cooled CCD camera and recorded on a desktop PC operating Simple PCI Imaging software (Hammamatsu Corporation, Japan). Lamp illumination, camera exposure and gain settings were strictly controlled to ensure that different samples could be compared.

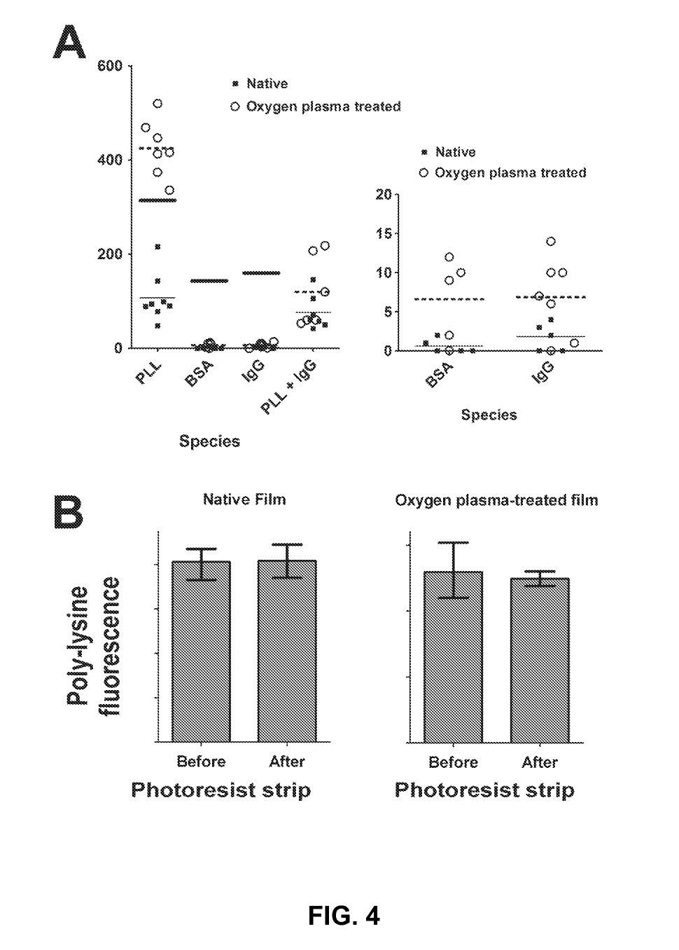

As shown in FIG. 4A, The PEO-like film permitted the adhesion of poly-lysine but not of BSA and IgG molecules. However, the presence of poly-lysine immobilized on the surface permitted the film to adsorb other molecules that it would otherwise be resistant to, such as IgG. (In the column labeled “PLL+IgG,” the pre-adsorbed poly-lysine was unlabeled, while the IgG was fluorescently tagged.) Treatment with oxygen plasma enhanced the adsorption of the poly-lysine to a level comparable or higher than on cell culture glass, while the adsorption of BSA and IgG only increased slightly. The right-hand panel is a close up of the BSA and IgG data plotted in the left-hand panel. On the charts, the thicker solid lines indicate the average level of adsorption on cell culture glass. The adsorption on glass provided a point of reference for each species, so that the adsorption of each on the PEO-like film relative to its adsorption on glass can be compared. The thinner lines indicate the average of the data points for native PEO-like film and the dotted lines represent the average of the data points for plasma oxidized film. Note: The fluorescence scale, vertical scale, is not the same for the left and right plots in FIG. 4A.

FIG. 4B illustrates that he adsorption of poly-lysine on both native (left) and oxygen plasma-treated films (right) was not measurably eroded by the photoresist stripping process.

### Example 7

### Fabrication of Micropatterned Surfaces

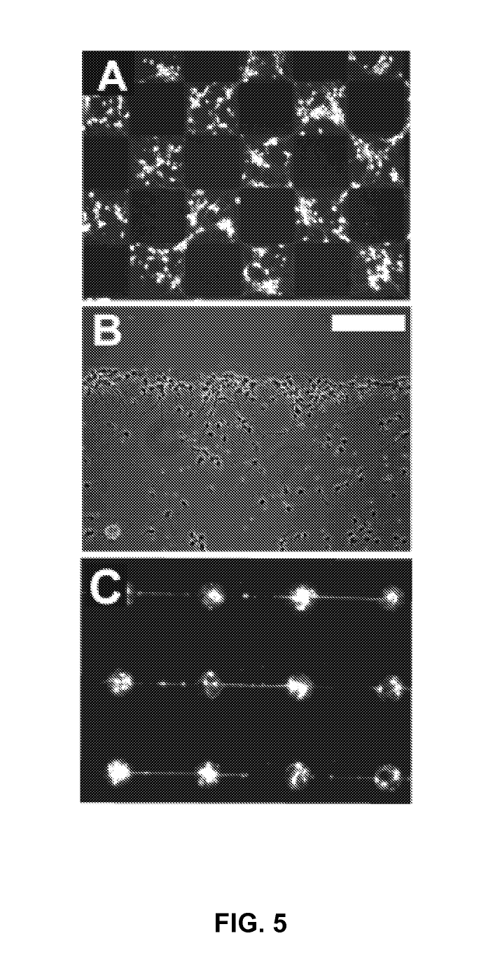

Fabrication of micropatterned surfaces. μ-Poly-Lysine-Adsorption-on-Cell-Repellant (μPLACeR) patterning process. To create the micropatterned surfaces, the PEO-like film was blanket deposited on 4-inch dia. Pyrex wafers. The film-covered wafer was then exposed for 1 min to vapors of HMDS to promote photoresist adhesion. (The wafer was not heated prior to this treatment.) A 1.3 micron layer of I-line positive photoresist (OiR 10i) (Arch Chemicals, Norwalk, Conn.) was spin coated on the wafer followed by a 90 sec. soft bake at 90° C. Desired patterns were then exposed on the wafer using a GCA 6200 wafer stepper (RZ Enterprises, Inc. Mountain View, Calif.), 10:1 reduction. The exposed pattern was developed with I-line developer (OPD 4262) (Arch Chemicals, Norwalk, Conn.) for 1 min, rinsed with DI water and blown dry. By using positive photoresist, areas that were intended to be cell adhesive were open and not covered by photoresist following the development step. These lithographically-developed substrates were subjected to a brief treatment of oxygen plasma, and then incubated with a 200 μg/mL solution of polyD-lysine (Sigma-Aldrich, 70,000-150,000 MW) in PBS (pH=7.2) for 1 hour, washed with DI water and then dried in air. This step coated the lithographically-defined, plasma oxidized regions of the PEO-like film with poly-lysine and rendered these regions cell adhesive, while the remaining areas were still cell repellant. Following this step, the remaining photoresist was removed by a 5-10 min. immersion in heated photoresist stripper (Baker PRS-3000) (J. T. Baker, Phillipsburg, N.J.) followed by 2 min. of sonication in the same stripper. The substrates were then thoroughly rinsed in distilled water and air-dried. The “lift-off” patterned poly-lysine areas remained and served as cell-adhesive regions, while the adjacent regions of the native PEO-like film, which were protected by photoresist and thus not coated with poly-lysine were cell repellant. FIGS. 5 A, B, and C provide examples of different micropatterns produced using this method.

### Example 8

### “Piggybacking” of Other Molecular Species

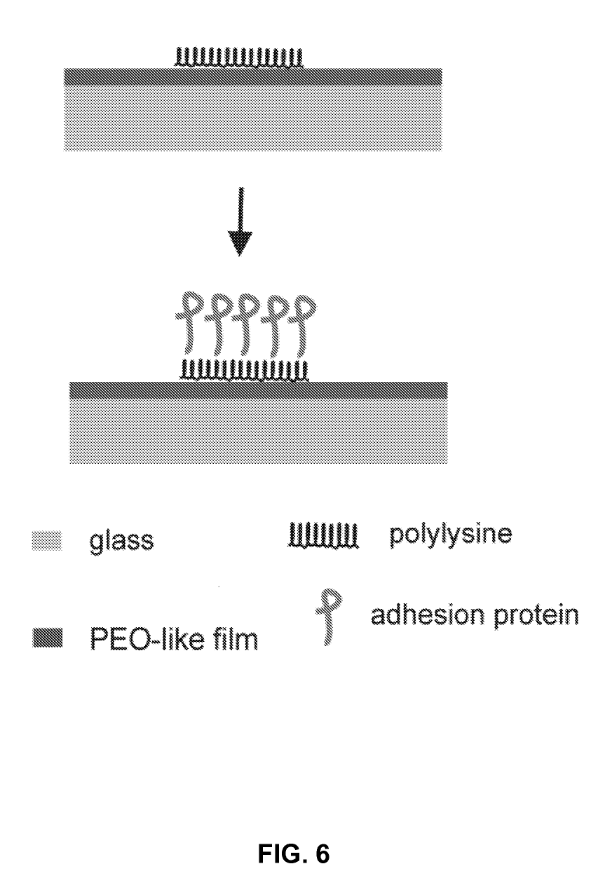

“Piggybacking” of other molecular species. While many molecular species do not adhere to any meaningful degree on the PEO-like film, the presence of pre-coated poly-lysine on lithographically-defined patterns on the film can mediate the immobilization of other bioactive proteins by their association with only the micropatterned poly-lysine. For example, a PEO-like film substrate with micropatterned poly-lysine can be incubated with a solution of laminin or IgG to immobilize these species on the same micropatterned regions. This simple “piggybacking” of other molecules of biological interest greatly extends the potential utility of the present micropatterning method. This application of the technology is diagrammed in FIG. 6.

### Example 9

### Neuron Cell Culture

Neuron cell culture. To evaluate the effectiveness of the micropatterned substrates for neuronal cell culture, primary hippocampal neurons from embryonic day 15 (E15) mice were plated onto the micropatterned substrates. The neurons were obtained using established protocols (Brewer, G. J. et al., J Neurosci Res, 35(5):567-76 (1993)). Briefly, hippocamppi were surgically removed from dissected brains of the E15 mice, and cells were isolated via tituration and enzymatic digestion. Cells were plated directly onto the micropatterned substrates and maintained in Neurobasal media (Invitrogen, Carlsbad, Calif.) supplemented with B27 (Invitrogen) and GlutaMAX (Invitrogen).

In addition to hippocampal neurons, retinal ganglion cells (RGC) obtained from 7-day-old mouse pups using established protocols (Barres, B. A., et al., Neuron, 1(9):791-803 (1988)) were also cultured on patterned substrates in which the extracellular matrix molecule laminin was immobilized onto poly-lysine patterns.

### Example 11

### Cell Viability and Compliance to Patterns

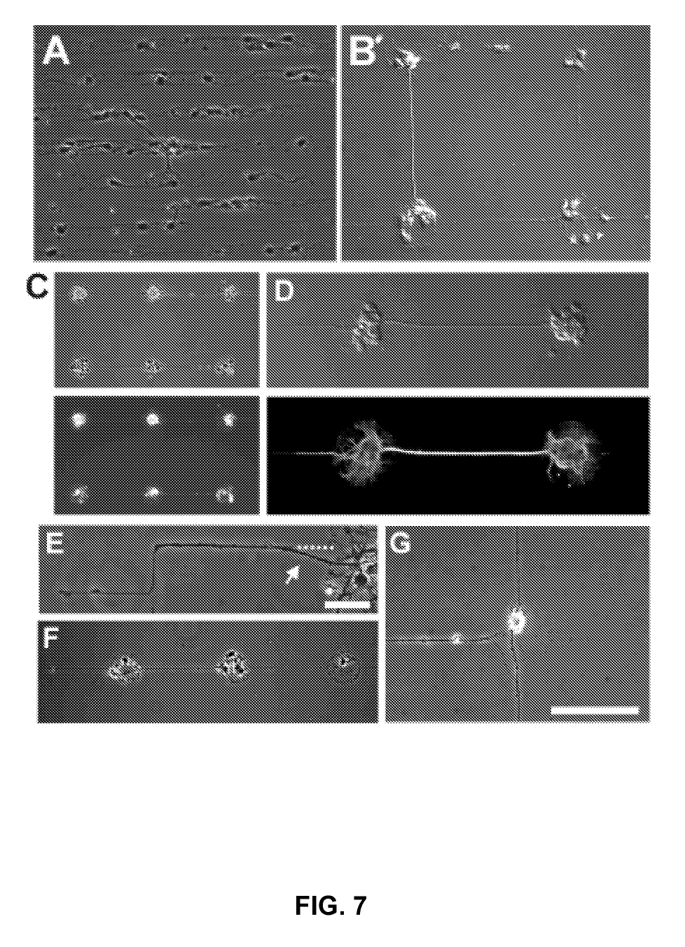

Cell viability and compliance to patterns. To quantitatively evaluate the viability of neurons cultured on the poly-lysine PEO-like films and the degree of cellular compliance to the patterned geometries, hippocampal neurons were plated on a checkered pattern, consisting of alternating 140×140 micron squares of cell adhesive (poly-lysine coated) and cell repellant (bare PEO-like film) regions. See FIG. 5A. To determine cell viability, cultures were stained with Fluo-4 AM calcium (Invitrogen) indicator dyes. Viable cells will fluoresce with an emission maximum of 525 nm as the calcium indicator is retained only in live cells after cleavage by esterases. Cell numbers on the adhesive regions were counted and compared to the number of cells on an equivalent area of the repellant region. To identify dead cells, cultures were stained with propidium iodide nucleic acid stain. Dead cells are permeable to this dye, which enters the dead cell and fluoresces upon association with nucleic acids. The compliance of neuronal attachment to adhesive regions was evaluated using the checkered patterns, on which cell bodies on both the cell adhesive and cell repellant squares were counted and compared. FIG. 5A. The compliance of neurite outgrowth along micropatterns was also evaluated along narrow 2 μm wide lanes of adhesive material. See FIG. 7B. For cells on micropatterns, an anti-tubulin antibody (anti-TUB 2.1, Sigma-Aldrich, St. Louis, Mo.) was used to stain intact microtubules using established protocols (Suh, L. H. et al., J Neurosci, 24(8):1976-86 (2004)).

### Results and Discussion

Film characterization. The PEO-like film, generated by plasma-induced polymerization of diglycol methyl ether, was deposited on planar substrates to serve as a non-fouling background to prevent cell attachment. To render this film as non-fouling as possible, the plasma power was kept minimal at around 1-2 W (Bretagnol, F. et al., Plasma Process Polym, 3:30-28 (2006); Forch, R. et al., Chem Vap Deposition, 13:280-294 (2007)). However, it noted that the process described herein used constant plasma power, as opposed to a pulsed delivery of plasma power. It is understood that pulsed delivery of power tends to reduce damage to the molecular structure of monomeric precursor, though both power formats have been successfully used for formation of PEO-like films (Bretagnol, F. et al., Plasma Process Polym, 3:30-28 (2006); Bretagnol, F. et al., Acta Biomater, 2(2):165-72 (2006); Forch, R. et al., Chem Vap Deposition, 13:280-294 (2007)). It is therefore possible that using low power can minimize molecular damage from ion bombardment, even under continuous power. While the molecular structure (i.e., degree of cross linking) of the film, which was deposited under continuous power was not characterized, the chemical composition was characterized to confirm that it could serve effectively as a cell repellant material in its native form. Concurrently, films whose surface properties had been tuned by plasma oxidation were likewise characterized to determine the resulting change in chemical composition associated with the enhanced adsorption of poly-lysine.

XPS analysis. As the first step in characterizing PEO-like film, the chemical composition of the deposited PEO-like film was determined using X-Ray Photoelectron Spectroscopy. By comparing the C1 and O1 peaks from the broad survey scan, it could be determined that the stoichiometric ratio of oxygen to carbon (O/C) was approximately 0.5, corresponding closely to the stoichiometry in the precursor molecule as well as polyethylene oxide itself. The high-resolution scan of the C1 peak, spanning 282 to 292 eV, revealed the contributions from the different types of carbon bonds (FIG. 2). This C1 spectrum consists of four peaks: a major component at 285 eV arising from C—C and C—H bonds; another important peak at 286.5 eV due to C—O bonds (ethers); and lesser peaks at 288 eV and 289.2 eV corresponding to C══O and O—C—O bonds and COOR(H) (esters and carboxyl) groups, respectively. Each high-resolution scan was fitted to these four peaks, and the individual contributions of each peak to the overall spectrum were determined from this fitting. For evaluating PEO-like character, the first two major components, corresponding to C—C/C—H and C—O moieties, respectively, and their relative intensities are the most essential factors. In the film, the peak corresponding to the C—O bonds, at 286.5 eV, accounted for around 65-70% of the intensity of the C1 peak, with most of the remaining fraction accounted for by the peak corresponding to the covalent C—C and C—H bonds, at 285 eV (FIG. 2A). This proportion implied that the film material had a PEO character ranging from 65-70% among three different samples. A small contribution from the C══O and O—C—O bonds, at 288 eV, was also present. Finally, contribution from the fourth component, representing ester and carboxyl groups (COOR(H)), at 289.2 eV, was negligible in the native film.

In previous work, it was determined that low power (˜1-2 W) plasma was the most desirable for creating a non-fouling film with chemistry and stoichiometry closely matching polyethylene oxide (Bretagnol, F. et al., Acta Biomater, 2(2):165-72 (2006)). By applying low plasma power in the PEO-like film generation and deposition recipe, the chemical characteristics of the film closely matched those of previously demonstrated, non-fouling films. With respect to the stoichiometric ratio of carbon to oxygen, and the relative proportion of carbon-based bonds, the film is chemically similar to non-fouling versions of the PEO-like material reported (for both pulsed and continuous) (Bretagnol, F. et al., Plasma Process Polym, 3:30-28 (2006); Bretagnol, F. et al., Sensors Actuators B, 123:283-292 (2007); Bretagnol, F. et al., Acta Biomater, 2(2):165-72 (2006); Sardella, E. et al., Plasma Process Polym, 1:63-72 (2004)). Previous work has shown that material of this chemical composition resists most protein adsorption and strongly resists cell attachment, rendering this PEO-like film an appropriate selection as a cell repellant background for the cell patterning method.

Film samples treated with oxygen plasma were also analyzed under XPS (FIG. 2B). Since the XPS measurements are derived from 10-20 nm depths within materials, it was difficult to precisely quantify changes at the very surface. Nevertheless, it was found that the brief oxygen plasma treatment slightly diminished the apparent PEO character of the film from ˜65-70% to ˜55%, while the oxygen to carbon ratio (O/C) remained at around 0.5. The decrease in PEO character was accompanied by a substantial increase of the C1 peak at 289.2 eV (to contributing about 7% of the C1 peak), indicating a marked increase in the proportion of COOR(H) (ester and carboxyl) groups (FIG. 2B, arrow). While the non-fouling nature of the PEO-like material has been attributed to the prevalence of ether bonds (C—O—C), the addition of ester and carboxyl groups to the surface tends to encourage the adsorption of species from aqueous solution (Bretagnol, F. et al., Sensors Actuators B, 123:283-292 (2007); Forch, R. et al., Chem Vap Deposition, 13:280-294 (2007)).

Contact angle. (Table 1) Surface hydrophilicity was characterized by contact angle measurements. Contact angle on native films averaged 59.7° (SD=1.8, n=36), which closely matched the PEO-like films reported previously. This contrasted with contact angle averages of 43.5° (SD=3.8, n=20) for the underlying polished Pyrex glass. Treatment with oxygen plasma, as described, resulted in modest initial decrease of the contact angle to around 43.9° (SD=2.6°, n=14). When exposed to air, the contact angle relaxed to 48.0° (SD=2.4°, n=12) after two hours, then to 52.7° (SD=3.9°, n=12) after two days, and finally to 57.0° (SD=2.7°, n=12) after four days. When the treated film was kept immersed in DI water at room temperature, the contact angle remained low and only relaxed to 47.4° (SD=1.9°, n=15) after four days. Plasma oxidation of polymeric materials such as poly-dimethylsiloxane (PDMS) has been widely applied in various applications to render surfaces more hydrophilic via the addition of oxygen-containing surface groups. Specifically, it is believed that the exposure to reactive oxygen ions results in the addition hydroxyl groups along the surface, imparting the surface with more negative charge (Chen, I. J. and Lindner, E., Langmuir, 23(6):3118-22 (2007); Ginn, B. and Steinbock, O., Langmuir, 19:8117-8118 (2003)). However, it has also been well documented that these changes in surface characteristics reverse when exposed to ambient atmospheric conditions either through conformational changes of the polymeric chains at the surface or migration of oligomers from the bulk to the surface. Similar mechanisms may be taking place within the PEO film, although the phenomenon for this material remains to be explicitly investigated.

TABLE 1
Contact angle (SD) (deg.) of the native and plasma treated films.

| | | Oxygen Plasma Treated | | | | |
|---|---|---|---|---|---|---|
| | Native | Immediate | 2 hr. in Air | 2 days in Air | 4 days in Air | 4 days in Water |
| Contact Angle | 59.7 (1.8) | 43.9 (2.6) | 48.0 (2.4) | 52.7 (3.9) | 57.0 (2.7) | 47.4 (1.9) |
| n | 36 | 14 | 12 | 12 | 12 | 15 |

Surface roughness. AFM measurements were performed in tapping mode along the surface of the native film with a cantilever tip (FIG. 3). Scanning was performed within 5 μm×5 μm areas at four random locations on the film surface (FIG. 3A). The deposited film was found to be smooth within a 2 nm range (FIG. 3A, B), too small to exert any topographical influences on cell attachment and behavior. This surface smoothness was unchanged after the brief plasma oxidation (FIG. 3B). This result confirms that the change in contact angle arising from the brief plasma treatment can be attributed predominately to change of surface chemistry and not to physical topography.

Deposition Rate. AFM measurements were also used to determine thickness of deposited films. Measurements indicated that a thickness of 31 nm was obtained with a deposition time of 35 min. under the described processing conditions, corresponding to a deposition rate of nearly 0.9 nm/min. This information was used to guide film deposition on process wafers, and a film thickness of around 15-25 nm was shown to mechanically withstand all of the subsequent photolithographic processes.

Protein adsorption. Although plasma polymerized, PEO-like films are generally considered to be non-fouling, few studies have explicitly evaluated the adsorptivities of various species from solution on the film's surface, and limited data is available primarily for BSA. It was sought to evaluate the adsorption not only of BSA but also the adsorption of poly-lysine and IgG, molecules, which are commonly used in cell culture. Poly-lysine in particular is a positively charged molecule that has a widely known tendency to adsorb to many types of surfaces. Adsorption on surfaces of the native and plasma tuned PEO-like film were compared.

Quantifying direct adsorption. Glass substrates on which the PEO-like film was blanket deposited were incubated with fluorescently labeled versions of poly-lysine, BSA, and IgG. These incubation tests showed that poly-lysine adsorbed to the native film, though to an extent less than on plain cell culture glass (FIG. 4A). In contrast, BSA and IgG did not appreciably adsorb to the PEO-like film, as their fluorescent signal remained close to the background level and was much less than their respective adsorptions on plain glass. PEO-like film substrates that had been treated with oxygen plasma (20 W for 15 sec. at ˜1.3 T) immediately prior to the incubation showed a marked increase in the adsorption of poly-lysine, even exceeding the adsorption of this species on plain cell culture glass. However, the adsorption of the BSA and IgG was only slightly increased (FIG. 4A, right panel). It is postulated that due to the positive charge of poly-lysine, the increase in adsorption of this species was due to an increase in negative charge-bearing moieties on the surface of plasma-oxidized film. As with many materials, even a brief exposure to oxygen plasma, hydroxyl groups will be added to the surface, transiently increasing the density of negatively charge, which can promote more adsorption of poly-lysine (Chen, I. J. and Lindner, E., Langmuir, 23(6):3118-22 (2007); Ginn, B. and Steinbock, O., Langmuir, 19:8117-8118 (2003); Belegrinou, S. et al., J Phys Chem B, 111(30):8713-6 (2007); Barbier, V. et al., Langmuir, 22(12):5230-2 (2006); Wu, Z. et al., Electrophoresis, 23(5):782-90 (2002)). Indeed, the XPS analysis of the PEO-like material is showed a marked increase in carboxyl and ester groups on the plasma oxidized surfaces, which was accompanied by a change in surface energy as seen in the change in the decrease in water contact angles. Previous studies have in fact shown that surface charge and wetability do have a significant influence the adsorption of molecules to surfaces (Burns, N. and Holmberg, K., Progr Colloid Polym Sci, 100:271-275 (1996)).

“Piggy-backing” on poly-lysine. Since it is a common practice to use a species like poly-lysine to facilitate immobilization of other bioactive molecules, the adsorption of poly-lysine for this PEO-like material was harnessed to bring about immobilization of other molecular species that would otherwise be largely repelled by the surface of the native film. As a demonstration, films with poly-lysine (unlabeled) were incubated in PBS solution then followed that with incubation with IgG-FITC in PBS. While IgG alone does not adsorb appreciably to the film surface, it adsorbs readily (FIG. 4A) onto surfaces that had been pre-coated with poly-lysine. Previous studies have demonstrated that surfaces coated with poly-lysine present fundamentally different apparent properties and exhibit different surface energies (Harnett, E. M. et al., Colloids Surf B Biointerfaces, 55(1):90-7 (2007)).

Surface patterning. Since poly-lysine adsorbed onto the surface of the PEO-like film, particularly after plasma oxidation of the surface, the following were developed: the μPLACeR process, a cellular micropatterning scheme that involves a single plasma-enhanced film deposition and a single photolithographic step to produce a substrate that simultaneously provided well-defined cell adhesive regions surrounded by adjacent, complementary areas that were cell repellant. The method of micropatterning involved the conventional spin coating of photoresist directly onto the PEO-like film and the application of standard photolithography on this substrate. The patterned photoresist served as the geometric template by which the poly-lysine immobilization was subsequently patterned by “lift-off,” creating patterns with resolution down to 1 micron (FIG. 1B). Following the adsorption of poly-lysine, the photoresist was completely stripped with the heated PRS-3000 stripper, leaving patterned cell adhesive regions coated with poly-lysine, and bare PEO-like film serving as cell repellant regions. This part of the PEO-like film remained physically and chemically unaltered throughout the photolithographic process, as indicated by XPS analysis of films that had undergone photoresist application, exposure, development and stripping (FIGS. 2C and D). Meanwhile, on the cell adhesive regions, the patterned poly-lysine on the surface of film was not affected by the photoresist stripping treatment, as there was no measurable erosion in the intensity of fluorescently labeled poly-lysine (FIG. 4B). This micropatterning process was easy to implement, and many copies of a patterned substrate were simultaneously produced.

Cell culture. Cellular Viability and Compliance on Patterned Substrates. To provide a more quantitative measure of the health of hippocampal neurons maintained on the patterned substrates, cell densities on these substrates were compared to densities on standard poly-lysine coated glass 3 days after cells were plated under identical conditions at ˜650 cell/mm². After 3 days, both substrates supported neurons with extensive neurite outgrowth and fasciculation. Cell densities on patterned substrates were similar to those on plain glass, and cell bodies and neurites faithfully followed the patterned geometries. Also, on both PEO-like film and conventional poly-lysine coated glass, a small number of dead cells stained by propidium iodide, could be observed interspersed with the live cells. These cells were small and spherical and, even under bright field, appeared distinct from living cells, whose cell bodies were flattened and spread out with multiple neurites extending. At day 3 there were an average of 471 (SD=98, n=8) cells/mm² on plain glass substrate coated with poly-lysine, while on the checkered pattern, a cell density of 952 (SD=264, n=12) cells per effective mm² of cell adhesive area (FIG. 5A). This higher cell density on checkered pattern is possibly attributable to the migration of neuronal cell bodies from cell repellant to cell adhesive areas during the initial period following cell plating, although such migrations have not been explicitly observed. Consistent with this interpretation, however, is the finding of local increases in neuronal density along edges of cell adhesive regions bordering cell repellant regions (FIG. 5B). These results indicate that micropatterns of poly-lysine deposited onto PEO-like films is a good substrate for neuronal attachment and growth.

Organizing Primary Neurons, Neurites, and Potential Neural Circuitry using Micropatterned PEO-like films. To quantitatively assess the degree of cellular and neurite compliance to the patterned substrates, hippocampal neurons, harvested from embryonic mice using standard protocol, were cultured on PEO-like films containing a variety of poly-lysine micropatterns. Within just one hour of plating, the association of neurons will cell adhesive patterns were already apparent. Cell bodies began to adhere almost immediately to poly-lysine coated areas, just as on poly-lysine coated glass typically used in conventional neuronal cell culture. Regions of bare PEO-like film were completely cell repellant to hippocampal neurons, and no adhesion of cells to this surface were observed. Compliance to the desired patterns as determined by counting the number of cells attached to the poly-lysine regions compared to the number of cells attached to an equally sized region of bare PEO-like film. The results showed that a very high degree of cellular compliance was achieved by the current micropatterning protocol. On 12 different samples, 3473 neurons were counted on cell adhesive poly-lysine containing regions, while only 3 neurons were found to be located in nominally cell repellant regions.

To assess the compliance of neurite extension on cell adhesive regions, 2 μm wide lanes of poly-lysine were patterned to serve as conduits to guide axonal and dendritic outgrowth. While in the initial 1-2 days after cell plating, cell bodies can be observed to adhere weakly to these 2 μm lanes, these neuron cell bodies subsequently detached over the course of two days. In contrast, the slender neurites extended along the narrow lanes, faithfully following the trajectory of these lanes (FIGS. 5C, 7B-F), including curved lanes. There were a few exceptions to this compliance at sharp bends, where neurites often appeared to “cut the corners.” It is believed that this reflects the fact that axons and dendrites do not adhere to their substrates continuously along their length but only at periodic locations where they develop adherent protein complexes.

Since neurons communicate with one another via their axonal processes, a potential use of neuronal micropatterning is the creation of well-organized neural circuitry on device surfaces. A commonly used geometry for patterning neurons is a square lattice configuration in which narrow lanes intersect at 90-degree angles. At these intersections, widened, circular cell adhesive regions are patterned to allow cell bodies to comfortably adhere, while neurites run along the interconnecting, narrow lanes. This standard configuration was applied with the patterning scheme, and found that the neuronal cell bodies and neurites complied with this simple circuit geometry (FIGS. 5C, 7B and 7C).

Compatibility with conventional immunodetection methods and fluorescence optical imaging. An important requirement for a versatile cell micropatterning method for biomedical research and perhaps for use in devices as well is compatibility with conventional cell function characterization. Glass substrates containing poly-lysine patterns deposited onto PEO-like films permit immunodetection of cellular constituents using conventional antibody immunostaining methods typically used for cell culture. Furthermore, neurons and axons grown on micropatterned substrates can be observed using standard optical microscopy that is widely available in research laboratories (FIG. 7D).

Shelf life. Another advantage to this current scheme is the persistence of biologically active micropatterns in ambient conditions. Substrates with micropatterned PEO-like films have been left at room temperature conditions for over one month and were subsequently found to elicit high compliance attachment and neurite outgrowth from hippocampal neurons (FIG. 5F). With most other techniques, patterned substrates must be used within a few days of preparation. Molecular monolayers in particular can degrade quickly after they are assembled on a substrate and are often subject to hydrolysis in aqueous environment.

Cell culture of primary neurons using “piggy-back” molecular patterning. While the micropatterned poly-lysine can be used directly to culture many types of neurons, other neuron types frequently require the presence of specific bioactive adhesion molecules to mediate attachment, survival, and neurite extension on a culture substrate. It was demonstrated (see FIGS. 4 A,B) that poly-lysine adsorbed on the PEO-like film facilitated the immobilization of other molecules that would not otherwise adhere to the film. Laminin, an important component of the extracellular matrix, was applied to a substrate with patterned poly-lysine. Laminin only adhered to the poly-lysine coated regions and not on the bare film. Subsequently, when retinal ganglion cells (RGC) (Barres, B. A. et al., Neuron, 1(9):791-803 (1988)), which require laminin for adhesion (Leng, T. et al., Invest Ophthalmol Vis Sci, 45(11):4132-7 (2004); Lindsey, J. D. and Weinreb, R. N., Invest Ophthalmol Vis Sci, 35(10):3640-8 (1994)), were plated on these substrates, cell bodies only adhered along patterned regions, and neurites within the 2 μm lanes faithfully followed the lanes' trajectories (FIG. 7G). No cells or neurites were found in the nominally cell repellant areas. While the immobilization of laminin was not explicitly quantified, these results are consistent with the “piggybacking” of laminin along the pre-patterned poly-lysine and demonstrated the utility of the present micropatterning scheme as a platform for the simple microscale immobilization of a variety of biologically relevant molecules.

Advantages of the micropatterning method. The μPLACeR, (μ-Poly-Lysine Adsorption on Cell Repellant) micropatterning scheme is superior to other conventional approaches to neuron and neurite patterning in several key respects. The scheme combines both cell adhesive and cell repellant regions side-by-side on a culture substrate to produce a high compliance of neuron cultures for a variety of configurations. By comparison, conventional patterning techniques have not produced the same high compliance and must often contend with cells taking hold within regions outside of the desired patterns. Micro-contact printing, for example, often does not provide an explicitly cell repellant material to help enforce compliance, though more recent developments have incorporated such provisions. Methods that provide that enforcement via cell-resistant molecular monolayers, such as those based on poly-ethylene-glycol (PEG), still exhibit a lesser degree of cellular compliance to the desired patterns (Corey, J. M. et al., IEEE Trans Biomed Eng, 43(9):944-55 (1996); Chang, J. C. and Wheeler, B. C., Pattern Technologies for Structuring Neuronal Networks on MEAs. In Advances in Network Electrophysiology, Taketani, M. and Baudry, M., Eds., Springer US, 153-189 (2006); Corey, J. M. and Feldman, E. L., Exp Neurol, 184 Suppl 1, S89-96 (2003)). This is due to the fragility of molecular monolayers and the difficulty in producing close-packed and continuous coverage over an entire surface. In contrast, the plasma-polymerized films are robust material—usually many molecules deep—that reliably provide continuous coverage and in the case of the PEO-like material, is highly resistant to cell attachment and adsorption of many molecular species.

While the μPLACeR scheme is not the first application of these plasma-polymerized PEO-like film for patterning cell position and growth, it is much easier to implement compared with previously reported schemes and appears to be the only use of this material for neuron patterning. Strategies for using plasma polymerized films have focused on creating adjacent patterns of cell repellant and cell adhesive surface on the same substrates; this has included the direct patterning of the film deposition (Henein, Y. et al., Sens Actuat B, 81:49-54 (2001); Pan, Y. et al., Plasma Polymers, 7(2):171-183) (2002)), combining different film materials side by side (Bretagnol, F. et al., Plasma Process Polym, 3:30-28 (2006); Sardella, E. et al., Plasma Process Polym, 1:63-72 (2004)), and selectively altering, or tuning, surface properties on desired patterns (Bretagnol, F. et al., Sensors Actuators B, 123:283-292 (2007); Bretagnol, F. et al., Nanotech, 19:125306 (2008)). To pattern bioactive molecules on PEO-like films, micro Contact Printing (μCP) has been used successfully to stamp a variety of cell adhesion species onto this material. This dependence on μCP to deliver these molecules is due to the highly non-fouling nature of these materials, which are widely recognized to resist adsorption of molecular species from aqueous solutions but appear to accept these species readily when dry (Henein, Y. et al., Sens Actuat B, 81:49-54 (2001); Pan, Y. et al., Plasma Polymers, 7(2):171-183 (2002); Ruiz, A. et al., Microelectr Engin, 84:1733-1736 (2007)). However, it has been established that species such as poly-lysine can adsorb to these PEO-like materials from aqueous solution. The present scheme therefore exploits and enhances this previously overlooked tendency of the plasma-polymerized PEO-like films. This use of adsorbed poly-lysine in solution is not merely easier to implement than μCP, but can be used to produce robust, high-resolution, cell adhesive patterns on the PEO-like film and in high volume (as in wafer scale production). In addition to serving as a direct as a molecular substrate for cell culture, poly-lysine can also be used as a foundation to immobilize additional molecular species that can then support the growth of more specialized populations of neurons.

Embodiments of the invention provide a simple yet robust technique for creating high-resolution organization and micropatterning of neurons and their cellular processes in culture. The μPLACeR technique uses a non-fouling, poly-ethylene oxide (PEO)-like film as a background material for a cell repellant culture substrate. The plasma polymerized PEO-like film confers several important advantages for patterning. The film can completely cover a substrate. It is robust and stable in both ambient air and in aqueous solutions. As a non-fouling material, it is highly cell-repellant, and when blanket deposited, renders the culture background highly resistant to cell attachment. Nevertheless, despite its non-fouling character, the material does selectively adsorb poly-lysine, a positively charged molecule that is widely used for mediating cell adhesion to substrates (West, J. K. et al., J Biomed Mater Res, 37(4):585-91 (1997)). With subtle tuning of the surface chemistry of this film via plasma oxidation, this adsorption can be greatly enhanced even though the film's non-fouling properties with respect to other molecular species are only slightly diminished. Based on this interaction between poly-lysine and PEO-like films, a micropatterning scheme for neuronal and other cell culture involving a single plasma-enhanced, film deposition step was developed, along with a single photolithographic step to create high-resolution, cell adhesive micropatterns of poly-lysine set against a cell repellant background. Primary neurons maintained on substrates patterned with this method were healthy and complied nearly perfectly with the lithographically defined patterns, and neurite growth remained restricted to narrow lanes, demonstrating that the patterning technique is robust and reliable. Moreover, the patterned substrates themselves could be stored for extended periods in ambient conditions without noticeable degradation in biological activity or cellular compliance to the micropatterns. This versatile micropatterning technique can be readily adapted for many applications including the creation of simple neural circuits and can be easily integrated with fabrication methods for various biomedical microdevices and biosensors. The μPLACeR patterning technique can be applied to other cell types as well.

### Example 12

### Micropatterned Culture of Fibroblasts

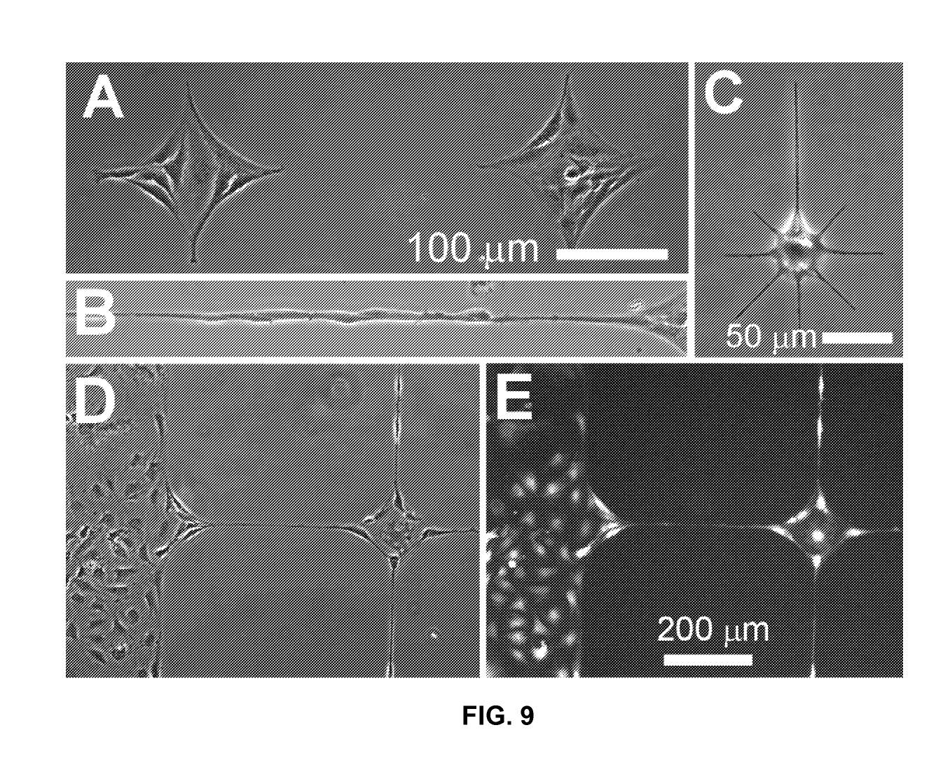

To demonstrate the versatility of the micropatterned substrates beyond neurons, 3T3 fibroblasts were cultured on the micropatterned surfaces of the present invention using DMEM media (Invitrogen) and Fetal Bovine Serum (UCSF Cell Culture Facility). Cultured cells proliferated and conformed to various micropatterned configurations with high compliance and high viability. FIG. 9 A-C shows examples of test patterns on which fibroblasts were successfully patterned along with the scale bars. The high viability of fibroblasts micropatterned on these substrates is shown in FIGS. 9 D&E, which show the same field of confluent cells in brightfield illumination (D) and fluorescence (E). In fluorescence view, cells were pre-loaded with a calcium-sensitive dye (Calcein AM, Invitrogen), which is only illuminated in living cells.

### Example 13

### Lone-Term Neuronal Culture on Micropatterned Substrates

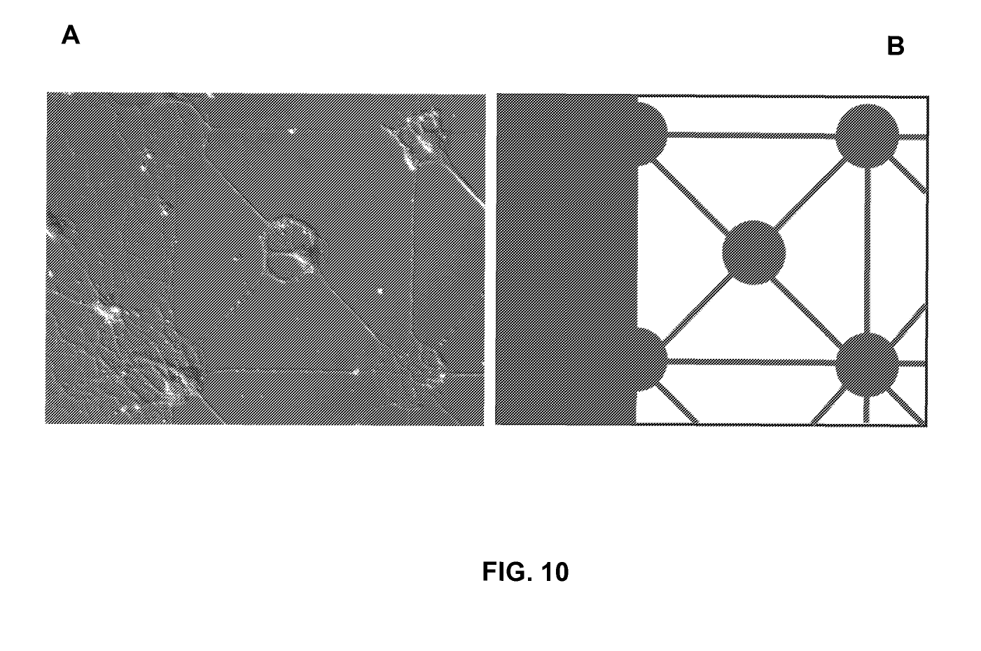

Neurons micropatterned on substrates of the present invention can be maintained viably and with high compliance to desired micropatterns. FIG. 10A shows 75 μm diameter, cell adhesive circles connected by a network of narrow (2 μm) cell-adhesive lanes. After 23 days in culture, cell bodies are stably maintained in the circular regions, while only the axons project along the lanes. A schematic of the micropattern is shown in FIG. 10B, with shaded areas being cell adhesive.

Any one or more features of one or more embodiments may be combined with one or more features of any other embodiment without departing from the scope of the invention.

All references, issued patents and patent applications cited within the body of the instant specification are hereby incorporated by reference in their entirety, for all purposes.

While the invention has been particularly shown and described with reference to a preferred embodiment and various alternate embodiments, it will be understood by persons skilled in the relevant art that various changes in form and details can be made therein without departing from the spirit and scope of the invention. Thus, the above description is illustrative but not restrictive. Many variations of the invention will become apparent to those skilled in the art upon review of the disclosure. The scope of the invention should, therefore, be determined not with reference to the above description, but instead should be determined with reference to the pending claims along with their full scope or equivalents.

1. A method, comprising:
depositing a cell-repellant film on a substrate;
masking a region of the cell-repellant film or the substrate;
modifying the masked region of the cell-repellent film or the substrate; and
depositing a cell-adhesive material on the modified region of the cell-repellant film or the substrate.

2-14. (canceled)

15. A composite structure, comprising:
a substrate;
a cell-repellant film deposited on the substrate, wherein one or both of the cell-repellant film and the substrate comprise a modified region; and
a cell-adhesive material adsorbed to the modified region.

16. The composite structure of claim 15, wherein the cell-repellant film comprises CH3-O—(CH2-CH2-O)n-CH3, wherein n is an integer from 1 to 7.

17. The composite structure of claim 15, wherein the cell-repellant film is produced using a plasma-enhanced chemical vapor deposition process.

18. The composite structure of claim 16, wherein the modified region of the cell-repellant film comprises a chemical modification caused by exposure to an oxidizing agent.

19. The composite structure of claim 18, wherein the oxidizing agent is an oxygen plasma.

20. The composite structure of claim 18, wherein the chemical modification comprises the presence of a carboxylate group, an ester group or combinations thereof.

21. The composite structure of claim 15, wherein the cell-adhesive material is a monolayer physisorbed onto the modified region of the cell-repellant film.

22. The composite structure of claim 21, wherein the cell-adhesive material comprises a polycationic molecule.

23. The composite structure of claim 22, wherein the polycationic molecule is poly-lysine, or poly-ornithine.

24. The composite structure of claim 22, further comprising a polypeptide adsorbed to the cell-adhesive material.

25. The composite structure of claim 24, wherein the polypeptide is an immunoglobulin, a serum albumin, or a laminin.

26. The composite structure of claim 21, wherein the cell-adhesive material comprises a predetermined pattern of features.

27. The composite structure of claim 26, wherein the predetermined pattern of features comprises feature elements having a dimension in the range of 1 μm to 100 μm.

28. The composite structure of claim 26, wherein the predetermined pattern of features comprises feature elements having a dimension in the range of 1 μm to 10 μm.

29. The composite structure of claim 26, wherein the predetermined pattern of features comprises feature elements having a dimension in the range of 1 μm to 5 μm.

30. The composite structure of claim 15, further comprising cells adherent to the cell-adhesive material.

31. The composite structure of claim 30, wherein the cells comprise fibroblasts, retinal ganglion cells, or hippocampal neurons.

32. The composite structure of claim 30, wherein the cells comprise neuronal cells.

33. The composite structure of claim 32, wherein the neuronal cells form a synapse.

34. (canceled)

35. (canceled)

36. (canceled)

37. (canceled)

38. (canceled)

39. (canceled)

40. (canceled)

41. (canceled)

42. (canceled)

43. The composite structure of claim 26, wherein the predetermined pattern of features is stable for at least two months when stored at 20° C. and 50% relative humidity.

44. The composite structure of claim 26, wherein the predetermined pattern of features is stable for at least twenty-one days when held at 37° C. and immersed in a cell-culture medium.

45. A composite structure, comprising:
a substrate;
a cell-repellant film deposited on the substrate, wherein one or both of the cell-repellant film and the substrate comprise a modified region; and
a cell-adhesive material adsorbed to the modified region,
wherein the cell-repellant film is a polyethylene oxide-like film, wherein the modified region comprises a chemical modification caused by exposure to an oxygen plasma, wherein the cell-adhesive material comprises poly-lysine molecules adsorbed to the modified region, and further comprising neuronal cells adherent to the cell-adhesive material.
